(19) United States
(12) Patent Application Publication
(10) Pub. No.: US 2021/0290694 A1
(43) Pub. Date: Sep. 23, 2021

(54) ACIDITY NEUTRALIZING COMPOSITION

(71) Applicant: Biozone Lifesciences Pvt. Ltd., Ahmednagar (IN)

(72) Inventor: Ritika Oberoi, Ahmednagar (IN)

(73) Assignee: Biozone Lifesciences Pvt. Ltd., Ahmednagar (IN)

(21) Appl. No.: 17/070,922

(22) Filed: Oct. 15, 2020

(30) Foreign Application Priority Data
Mar. 21, 2020 (IN) 202021012313

(51) Int. Cl. A61K 35/742; A61K 36/064

(52) U.S. Cl. CPC A61K 35/742; A61K 36/064

(57) ABSTRACT

The present invention relates to an acidity neutralizing composition comprising, bacterium for decreasing somatic cell count, wherein the bacterium promotes growth of various bacteria, yeast for scavenging oxygen in rumen of the ruminant and dextrin mixed for boosting production of milk in ruminants and protect cattle and other ruminants from diseases.

### FIELD OF THE INVENTION

The present invention relates to cattle feed supplement for neutralizing acidity. More specifically, the present invention relates to organic composition for helping the animals by neutralizing the acidity in their stomach. Moreover, the invention also helps to increase the productivity of milk from animals.

### BACKGROUND OF THE INVENTION

The domestication of animals is an ancient practice that may results in symbiotic relation, safety, and moreover production of various things, but there are many health problem associated with them, since they graze and feed variety of food, it may result indigestion in animals. Due to indigestion and over feeding of carbohydrates, acidic balance inside the stomach of ruminants may get upset that result in ruminal acidosis.

Due to ruminal acidosis, cattle may become depressed, go off feed, have an elevated heart rate and diarrhea. The cattle graze large quantity of lush green legumes and fed with high amount of concentrates. The large amount of grazing results in bloating. Conventionally, various traditional methods were practiced to overcome this problem, where cowherd makes an insertion on the cattle's stomach with a sharp object to release the gas but this action may result in abscess and infection, this may also lead to miscarriage of pregnant cattle as the calf in the womb may hurt by the thrust of tapping or insertion of such sharp objects.

To solve this problem various medicines were introduced for improving the digestion in animals, but such medicines are not found to be very effective.

US20140130225A1 discloses about methods and compositions for the following: improving rumen fermentation efficiency; increasing the efficiency with which dietary starch is utilized while preventing a deleterious increase in ruminal lactic acid concentration and/or a deleterious drop in ruminal pH; and promoting the growth of beneficial ruminal microorganisms. As a result, there is a concurrent improvement in the productivity and profitability of ruminant animal husbandry, especially in the dairy and beef industries. These methods and compositions provide for the supplementation of the diet of a ruminant animal with exogenous amylase at relatively low levels, without any concomitant need for exogenous fibrolytic enzymes such as cellulases and xylanases. The methods and compositions of the present invention can further comprise supplementation with yeast preparations, rumen buffering agents, ionophores or other growth and productivity stimulating agents.

The prior art talks about digestion in animals but the disadvantage of prior art is that the invention only focus on digesting starch content that present in the animal's food. Another disadvantage of prior art is that the invention may only digest the food but cannot increase the immunity of animals.

JPH06237702A discloses about increasing the amino acid level in ruminants, thus enable the increased milk production as well as increased health and appetite by giving the ruminants a specific feed additive from their antenatal period to lactation period. At first, from 3 weeks before their delivery day, feed additive which contains lysine and/or methionine and is protected from their rumen stomach are given. Further, the feed supply is continued during their lactation period, for 5 months at most, to increase the amino acid level in the ruminants. The feed additive is obtained by coating the nuclei containing methionine and/or lysine with a coating agent containing lecithin, inorganic substances which are stable in neutral and soluble in acid, 14C-22C monocarboxylic acid, hardened vegetable oil, wax and the like. The inorganic substance is preferably magnesium carbonate.

The prior art discussed in above paragraph provides a feed additive that contains methionine. Methionine used for increasing production of milk, but the disadvantage of the prior art is that invention contain methionine that may cause headache, heartburn, nausea, diarrhea, dizziness, or drowsiness to cattle.

In order to provide a supplement for cattle to increase the quantity of milk by controlling the acidity produce in the stomach of cattle, there is a vital need to provide a composition to improve the digestion of cattle to boost the production of milk.

### Objects of the Invention

The principal object of the present invention is to overcome the disadvantages of the prior art.

An object of the present invention is to provide a composition for improving digestion of cattle.

Another object of the present invention is to provide a composition for improving the production of milk in animals.

Yet another object of the present invention is to provide a composition for increasing the immunity against various harmful diseases.

The foregoing and other objects, features, and advantages of the present invention will become readily apparent upon further review of the following detailed description of the preferred embodiment as illustrated in the accompanying drawings.

### SUMMARY OF THE INVENTION

The present invention discloses about cattle feed to eliminate the problem of indigestion in ruminants, by controlling the formation of acid in cattle's stomach. The invention increases the immunity of the cattle and boosts the milk production.

According to an embodiment of the present invention, an acidity neutralizing composition for ruminants comprising, bacterium added in the composition to decrease somatic cell count, wherein the bacterium promotes growth of various bacteria, yeast fused in the composition for scavenging oxygen in rumen of the ruminant, and dextrin mixed in the composition to boost production of milk in the ruminants and the composition also plays a vital role in improving the immune system and thus protect the ruminants from diseases.

While the invention has been described and shown with particular reference to the preferred embodiment, it will be apparent that variations might be possible that would fall within the scope of the present invention.

### DETAILED DESCRIPTION OF THE INVENTION

The following description includes the preferred best mode of one embodiment of the present invention. It will be clear from this description of the invention that the invention is not limited to these illustrated embodiments but that the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In any embodiment described herein, the open-ended terms “comprising,” “comprises,” and the like (which are synonymous with “including,” “having” and “characterized by”) may be replaced by the respective partially closed phrases “consisting essentially of,” consists essentially of,” and the like or the respective closed phrases “consisting of,” “consists of, the like.

As used herein, the singular forms “a,” “an,” and “the” designate both the singular and the plural, unless expressly stated to designate the singular only.

The word “ruminant” used in the invention is to denote the milk producing animals.

The present invention talks about composition for neutralizing acid in stomach of ruminants. The invention provides pro-biotic feed supplement to the ruminant that increases immunity to fight against various harmful diseases and boost the productivity of milk.

The contents used in making of acid neutralizing composition for the ruminants, wherein composition comprises, bacterium for decreasing somatic cell concentration. The addition of bacterium also promotes the growth of various bacteria like ruminal, proteolytic and amylolytic bacteria. The composition is mixed with different type of ruminant food to feed the ruminants.

The bacterium used in composition is Bacillus subtilis that may also known as Bacillus subtilis cells. The Bacillus subtilis cells are typically rod-shaped, commonly found in upper layers of the soil. The Bacillus subtilis is able to enter in a dormant form called endospore that is able to tolerate extreme environmental conditions.

The Bacillus subtilis used in the composition decreases somatic cell count in the ruminant's body. The Bacillus subtilis increases the quality of milk as it reduces the somatic cell count. The Somatic cell count is total number of cells per milliliter in ruminant's milk. The injuries or infection on a mammary gland of the ruminant results in clinical mastitis with varying degrees of visible signs of the clinical mastitis where no visible symptoms occur. Monitoring of somatic cell count is critical in diagnosing cattle with subclinical mastitis. The ruminant body needs to decrease the somatic cell count to improve the quality of milk.

After mixing Bacillus subtilis, yeast is added in composition. Yeast improves digestion and scavenges oxygen in rumen of the ruminants. The addition of yeast also enhances immune function and marks positive metabolic changes such as lowering body temperatures of dairy cows during heat stress. Dairy producers may often see practical benefits of yeast, particularly in lactating dairy barn improved feed intakes, greater feed efficiency, better feed resulting in optimum milk performance.

Saccharomyces cerevisiae is a species of yeast that used in the composition for scavenging of cattle food. Saccharomyces cerevisiae is originally isolated from a skin of grapes or may found on the skins of dark-colored plums fruit that exists among the waxes of the cuticle. Saccharomyces cerevisiae is also known by its more common names that is brewer's yeast or baker's yeast. Brewer's yeast used as a protein supplement, energy booster and immune enhancer.

The Saccharomyces cerevisiae is small with a short generation time that is 1.25-2 hours at 30° C. or 86° F. and easily cultured. The Saccharomyces cerevisiae is used as a pro-biotic supplement that is useful for prevention or treatment of several gastrointestinal diseases. The Saccharomyces cerevisiae also used to reduce risk of antibiotic-associated diarrhea in animals.

A hydrolyzed starch that is also known as dextrin is mixed in the composition to boost the production of milk in cattle. Basically the dextrin is made from various starches such as potato, corn, tapioca, rice, arrowroot or wheat. Wheat starch is a product from extracting gluten of wheat. The wheat starch is sprayed with acid while being agitated and then suspended in water by the acids or enzymes.

After maturing, the wheat starch is dried in a roaster where it is continuously heated and stirred. The dextrin taken out from the roaster and cooled after the conversion. The dextrin is humidified before packing to keep away from getting lumpy or foaming. Heating times and acids vary depending upon the manufacturer and the final product usage. Wheat dextrin improves animal body's absorption and retention of minerals and nutrients, while improving cattle's immune system. Chemical formula of dextrin is (C6 H10 O5)n, where ‘n’ represents the number of glucose molecules in carbon chain, IUPAC name of dextrin is 2-{[4,5-dihydroxy-2-(hydroxymethyl)-6-{[4,5,6-trihydroxy-2-(hydroxymethyl)oxan-3-yl]oxy}oxan-3-yl]oxy}-6-(hydroxymethyl)oxane-3,4,5-triol.

The dextrin used in the composition is in powder form. The dextrin is to boost the productivity of milk. The composition also provides immunity to the ruminants to fight against various harmful diseases like prussic acid poisoning, bloat, grass tetany and visceral pain.

The dextrin powder is prepared by heat treatment in presence of chemicals to suitably modify characteristics and properties of starch. Unlike starch, dextrin is soluble in water. Severity of the treatment determines a degree of solubility that is basis of classifying or grading dextrin. Finished dextrin is very fine powder varying in color from pure white to off white. The dextrin is prepared by roasting starch in the presence of acid that may chemically change the character and properties of starch.

The composition is made up of organic substances; this minimizes the side effects from the composition. The composition is mixed with different type of ruminant's food like seed meal, oil cake, bran powder and alike. A proper ratio is maintained to mix the composition with the ruminant's food for example: To 1.5 kg of concentrate 1.5 gm of composition is added. This blend of concentrate and composition are given to the ruminants two times a day. Apart from this 20 kg of fodder has to be given to the animal per day. This increases the productivity of milk up to 18% by controlling the formation of acid in the stomach of the ruminants.

The cowherd does not need to make insertion on cattle's stomach with sharp objects, for removing the gas during the bloating of stomach and the production of milk also increases by feeding the composition to the cattle. Moreover, the invention provides immunity to the cattle to fight against various diseases.

Although the field of the invention has been described herein with limited reference to specific embodiments, this description is not meant to be construed in a limiting sense. Various modifications of the disclosed embodiments, as well as alternate embodiments of the invention, will become apparent to persons skilled in the art upon reference to the description of the invention.

We claim:

1. An acidity neutralizing composition, comprising:
bacterium added in said composition to decrease somatic cell count, wherein said bacterium promotes growth of various other bacteria;
yeast fused in said composition for scavenging oxygen in rumen of said ruminant; and
dextrin mixed in said composition to boost production of milk in said ruminants.

2. The composition as claimed in claim 1, wherein said bacterium is Bacillus subtilis.

3. The composition as claimed in claim 1, wherein said yeast is preferably Saccharomyces cerevisiae.

4. The composition as claimed in claim 2, wherein said Bacillus subtilis helps in the proliferation of said bacteria that may include ruminal, proteolytic and amylolytic bacteria.

5. The composition as claimed in claim 1, wherein said dextrin may substitute by hydrolysis of starch.

6. The composition as claimed in claim 1, wherein said bacterium, yeast and dextrin is added 90, 750 and 8160 Kg respectively for making 9.0 ton of feed supplement, wherein substances in said composition are organic in nature to minimize side effect from said composition.

7. The composition as claimed in claim 1, wherein 18% of milk productivity increases by feeding said composition with said fodder.

8. The composition as claimed in claim 1, wherein said composition is mixed with different type of ruminant food like seed meal, oil cake, bran powder and alike, wherein said composition provides immunity to said ruminants to fight against various diseases that may include but not limited to prussic acid poisoning, bloat, grass tetany, visceral pain and alike.
